(19) United States
(12) Patent Application Publication
(10) Pub. No.: US 2026/0215759 A1
(43) Pub. Date: Jul. 30, 2026

(54) WIRELESS ULTRASOUND SCANNER WITH VARIABLE QUICK RESPONSE (QR) CODE

(71) Applicant: KONINKLIJKE PHILIPS N.V., EINDHOVEN (NL)

(72) Inventor: McKee Dunn Poland, Andover, MA (US)

(21) Appl. No.: 19/145,224

(22) PCT Filed: Dec. 28, 2023
(86) PCT No.: PCT/EP2023/087874

§ 371 Date: Jul. 2, 2025

Related U.S. Application Data
(60) Provisional application No. 63/437,179, filed on Jan. 5, 2023.

(51) Int. Cl. A61B 8/00; A61B 8/14

(52) U.S. Cl. CPC A61B 8/4438; A61B 8/14; A61B 8/4427; A61B 8/4461; A61B 8/4472; A61B 8/461; A61B 8/565

(57) ABSTRACT

An ultrasound scanner is provided for acquiring ultrasound images of a subject. The ultrasound scanner includes a handheld housing (111); a transducer array (112) arranged within the housing, where the transducer array includes multiple transducer elements configured to emit ultrasound signals into the subject, and to receive ultrasound echo signals responsive to the ultrasound signals being reflected in the subject; at least one processor (120) arranged within the housing, where the at least one processor is configured to obtain at least one parameter, determine content of a quick response (QR) code (128) based on the at least one parameter, and to generate the QR code, wherein the content of the QR code is variable; and a display (125) arranged on the housing, where the display is configured to render and display the QR code generated by the at least one processor, where the QR code is readable by a QR scanner of a host system.

### BACKGROUND

An ultrasound imaging system typically includes an ultrasound probe and a processing system. The ultrasound probe includes an array of ultrasound transducer elements configured to emit acoustic waves through a patient's body and to receive echo signals as the acoustic waves are reflected from the tissues, organs and other structures. The timing and strength of the echo signals generally correspond to the size, shape, and mass of the structures in the patient's body, images of which are displayed to a user of the ultrasound imaging system.

A wireless ultrasound probe wirelessly transmits electrical data from the ultrasound echo signals or ultrasound image data to a host system, depending on the extent of signal processing performed at the ultrasound probe. It is difficult, though, to seamlessly, quickly, and securely connect the wireless ultrasound probe to the host system, such as a tablet. A typical workflow includes activating a connection page on the host system, examining entries of nearby wireless ultrasound probes nearby in a menu on the connection page, and selecting one of the wireless ultrasound probes to connect to. This process requires some way of unambiguously identifying the wireless ultrasound probes. Conventional systems may assist in the identification process by flashing confirmation lights on the wireless ultrasound probe, for example, when the connection is made. However, the identification process is cumbersome and does not inspire confidence about having selected the correct wireless ultrasound probe, especially when there are multiple wireless systems in the vicinity doing the same thing.

Selecting the wrong ultrasound probe results in at least wasted time, and may also compromise patient privacy if the error is not discovered immediately. When the selected ultrasound probe is not itself verified to be authentic or is not the intended selection (even if it is a valid) before the wireless connection is established, then ultrasound scan data and/or other sensitive patient data generated by the system may be transferred through the host system to the wrong database. That is, once wirelessly connected, sensitive information originating from the ultrasound probe may be sent to the host system. The possibility of sending sensitive information is especially prevalent when such information may be entered directly into the ultrasound probe, e.g., through a user interface located on the probe.

### SUMMARY

According to a representative embodiment, an ultrasound scanner is provided for acquiring ultrasound images of a subject. The ultrasound scanner includes a handheld housing; a transducer array arranged within the housing, where the transducer array includes multiple transducer elements configured to emit ultrasound signals into the subject, and to receive ultrasound echo signals responsive to the ultrasound signals being reflected in the subject; at least one processor arranged within the housing, where the at least one processor is configured to obtain at least one parameter, determine content of a quick response (QR) code based on the at least one parameter, and to generate the QR code, where the content of the QR code is variable; and a display arranged on the housing, where the display is configured to render and display the QR code generated by the at least one processor, where the QR code is readable by a QR scanner of a host system.

According to another representative embodiment, a method is provided for connecting a wireless ultrasound scanner with a host system for performing ultrasound imaging of a subject. The method includes receiving at least one parameter providing information related to the ultrasound imaging; determining content of a QR code based on the at least one parameter, where the content of the QR code is variable; generating the QR code to include the determined content; rendering and displaying the QR code in a display arranged on the ultrasound scanner; and reading the QR code at the host system using a QR scanner to retrieve the content of the QR code, enabling performance of the ultrasound imaging of the subject.

According to another representative embodiment, an ultrasound scanner is provided for acquiring ultrasound images of a subject. The ultrasound scanner includes a handheld housing; a transducer array arranged within the housing, where the transducer array includes multiple transducer elements configured to emit ultrasound signals into the subject, and to receive ultrasound echo signals responsive to the ultrasound signals being reflected in the subject; a transceiver configured to establish wireless communication with a host system via a wireless communication link; at least one processor arranged within the housing, where the at least one processor is configured to obtain configuration information of the wireless communication link for establishing wireless communication with the host system, to determine content of a QR code based on the configuration information, and to generate the QR code, where the content of the QR code is variable; and a display arranged on the housing, where the display is configured to render and display the QR code generated by the at least one processor, where the QR code is readable by a QR scanner of the host system to establish the wireless communication.

### BRIEF DESCRIPTION OF THE DRAWINGS

The representative embodiments are best understood from the following detailed description when read with the accompanying drawing figures. It is emphasized that the various features are not necessarily drawn to scale. In fact, the dimensions may be arbitrarily increased or decreased for clarity of discussion. Wherever applicable and practical, like reference numerals refer to like elements.

FIG. 1 is a simplified schematic diagram of an ultrasound imaging system including a wireless ultrasound scanner and a host system, according to a representative embodiment.

FIG. 2 is a simplified block diagram of the wireless ultrasound scanner in the ultrasound imaging system, according to a representative embodiment.

FIG. 3 is a simplified block diagram of the host system in the ultrasound imaging system, according to a representative embodiment.

FIG. 4 is a simplified schematic diagram of a representative processor circuit implemented by the ultrasound imaging system, according to a representative embodiment.

FIG. 5 is a flow diagram of a method of providing a QR code for controlling an ultrasound scanner, according to a representative embodiment.

### DETAILED DESCRIPTION

In the following detailed description, for the purposes of explanation and not limitation, representative embodiments disclosing specific details are set forth in order to provide a thorough understanding of an embodiment according to the present teachings. Descriptions of known systems, devices, materials, methods of operation and methods of manufacture may be omitted so as to avoid obscuring the description of the representative embodiments. Nonetheless, systems, devices, materials and methods that are within the purview of one of ordinary skill in the art are within the scope of the present teachings and may be used in accordance with the representative embodiments. It is to be understood that the terminology used herein is for purposes of describing particular embodiments only and is not intended to be limiting. The defined terms are in addition to the technical and scientific meanings of the defined terms as commonly understood and accepted in the technical field of the present teachings.

It will be understood that, although the terms first, second, third, etc. may be used herein to describe various elements or components, these elements or components should not be limited by these terms. These terms are only used to distinguish one element or component from another element or component. Thus, a first element or component discussed below could be termed a second element or component without departing from the teachings of the inventive concept.

The terminology used herein is for purposes of describing particular embodiments only and is not intended to be limiting. As used in the specification and appended claims, the singular forms of terms “a,” “an” and “the” are intended to include both singular and plural forms, unless the context clearly dictates otherwise. Additionally, the terms “comprises,” “comprising,” and/or similar terms specify the presence of stated features, elements, and/or components, but do not preclude the presence or addition of one or more other features, elements, components, and/or groups thereof. As used herein, the term “and/or” includes any and all combinations of one or more of the associated listed items.

As used in the specification and appended claims, and in addition to their ordinary meanings, the term “approximately” mean to with acceptable limits or degree. For example, “approximately 20 GHz” means one of ordinary skill in the art would consider the signal to be 20 GHz within reasonable measure.

As used in the specification and appended claims, in addition to their ordinary meanings, the term “substantially” means within acceptable limits or degree. For example, the “plurality of transducer ports are substantially the same” means one of ordinary skill in the art would consider the plurality of transducer ports to be the same.

According to various embodiments, a wireless ultrasound scanner system includes an onboard wireless probe and a companion host system that displays ultrasound images and measurements, and also allows control of the ultrasound scanner system as a whole.

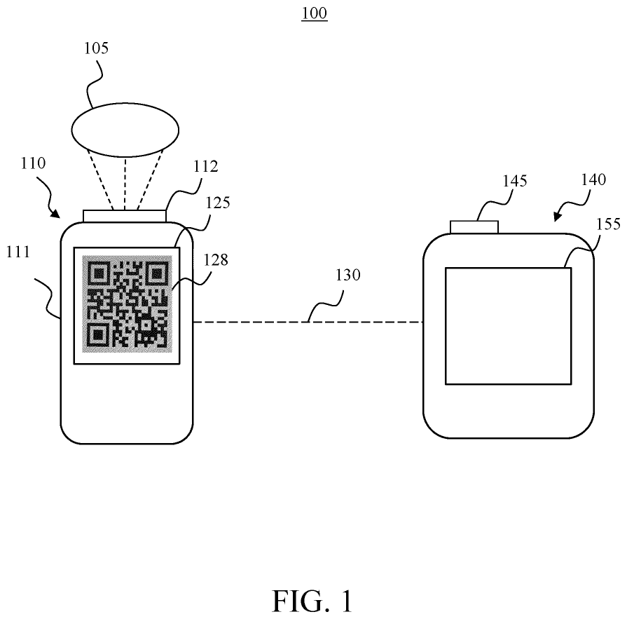

FIG. 1 is a simplified schematic diagram of an ultrasound imaging system including a wireless ultrasound scanner and a host system, according to a representative embodiment.

Referring to FIG. 1, ultrasound imaging system 100 includes a wireless ultrasound scanner 110 and a host system 140, which are in communication with one another over a wireless communication link 130, indicated by a dashed line. The wireless communication link 130 may be a WiFi link or other type of wireless link accessible through a wireless access point, for example, or may be a point-to-point wireless link with no intermediate access point, such as a Bluetooth link or a Bluetooth low energy (BLE) link, for example. Of course, any other type of suitable wireless communication links may be incorporated without departing from the scope of the present teachings.

The ultrasound scanner 110 includes a handheld housing 111 and a display 125 that is arranged on the housing 111. The display 125 may be a liquid crystal display (LCD), a light emitting diode (LED) display, a flat panel display, or a solid-state display, for example, although any type of compatible display may be incorporated without departing from the scope of the present teachings. The ultrasound scanner 110 also includes a transducer array 112, arranged within the housing 111, that includes multiple transducer elements configured to emit ultrasound signals into subject 105 (e.g., patient's body or anatomical structure), and to receive ultrasound echo signals responsive to the ultrasound signals being reflected in the subject 105 for performing ultrasound imaging examinations of the subject 105.

The display 125 is configured to render and display a variable quick response (QR) code 128. The content of the QR code 128 is determined by a processor also arranged within the housing 111 of the ultrasound scanner 110 based on at least one parameter, as discussed below. The QR code 128 provides information to the host system 140 relevant to the ultrasound imaging. For example, the information readable from the QR code 128 may include association information for making an association between the ultrasound scanner 110 and the host system 140. Since the QR code 128 is variable, it may contain changing association information, enabling use of the QR code 128 even when the association mechanism is adaptable relative to the administrative environment, such as the network infrastructure including the wireless communication link 130. For example, the association information may include configuration information for establishing the wireless communication between the ultrasound scanner 110 and the host system 140 over of the wireless communication link 130. When the wireless communication link 130 is through a WiFi network, for example, the configuration information provided by the QR code 128 may include a wireless IP address dynamically assigned by the wireless local area network (WLAN), a service set identifier (SSID), a password, and/or an authentication data packet of the ultrasound scanner 110. Other types of information that may be provided by the QR code 128 are discussed below.

The host system 140 includes a QR scanner 145 and a host display 155. The host system 140 may be implemented by a Samsung S7™ Android tablet or an Apple iPad Pro™ tablet, for example, although any compatible ultrasound host system may be incorporated without departing from the scope of the present teachings. The QR scanner 145 may be a camera, for example. The QR scanner 145 is configured to scan the QR code 128 displayed on the display 125 of the ultrasound scanner 110. In the depicted embodiment, the QR scanner 145, e.g., camera, is shown integrated with host system 140, which would require physically moving the host system 140 and/or the ultrasound scanner 110 to enable reading of the QR code 128. Alternatively, the QR scanner 145 may be moveable relative to the host system 140, via a wireless connection (e.g., Bluetooth) or a wired connection (e.g., cable or cord), so that the QR scanner 145 may be maneuvered independently of the host system 140 to read the QR code 128.

As discussed further below, the ultrasound scanner 110 is configured to perform the functionality of at least an ultrasound probe with regard to emitting ultrasound waves into the subject 105, receiving ultrasound echoes from the emitted ultrasound waves being reflected in the subject 105, and providing digitized echo signals corresponding to the ultrasound echoes. In this case, the ultrasound scanner 110 transmits the digitized electrical echo signals to the host system 140 via the wireless communication link 130. In some embodiments, though, the ultrasound scanner 110 is further configured to perform at least part of the functionality typically performed by the host system 140, including converting the digitized echo signals to ultrasound image data. In this case, the ultrasound scanner 110 is able to transmit the ultrasound image data to the host system 140 via the wireless communication link 130.

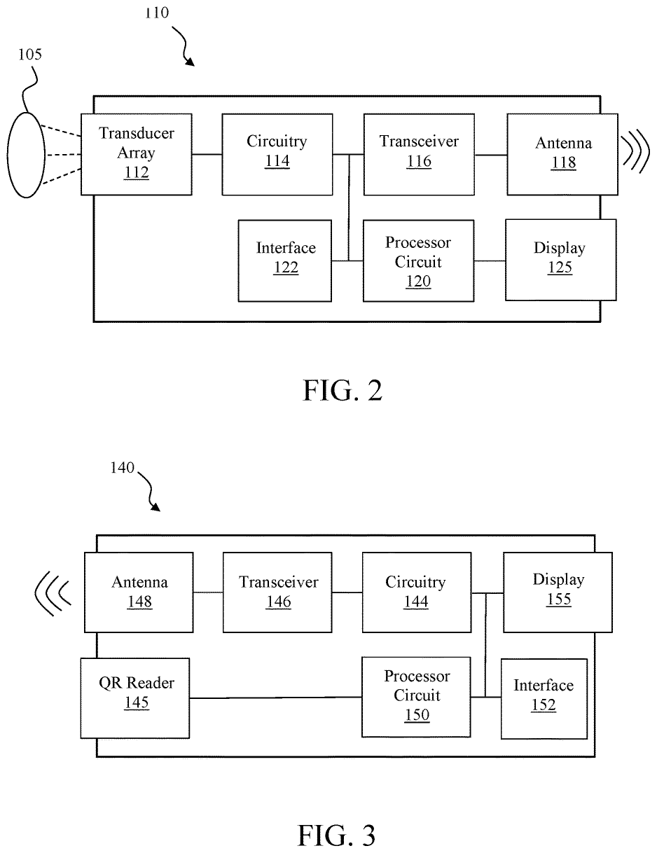

FIG. 2 is a simplified block diagram of the wireless ultrasound scanner in the ultrasound imaging system, according to a representative embodiment. At a high level, the ultrasound scanner 110 emits ultrasound waves towards the subject 105 and receives ultrasound echoes that are reflected from the subject 105. The ultrasound scanner 110 digitizes electrical echo signals representative of the received ultrasound echoes, and transmits the digitized signals as a digital echo data stream over the wireless communication link 130 to the host system 140 for processing and image display. The wireless communication link 130 is able to communicate data in analog format, digital format, and/or both analog and digital formats. The ultrasound scanner 110 may be in any suitable form of handheld device for imaging various body parts of the subject 105 while positioned outside of the body, such as a transthoracic echocardiography (TTE) probe, for example, where the housing 111 is configured to be grasped by the hand of a user (e.g., sonographer, physician or other clinician). Any of the components of the ultrasound scanner 110 shown in FIG. 2 may be positioned or stored in a housing 111.

In the depicted configuration, the ultrasound scanner 110 includes a transducer array 112, probe circuitry 114, a transceiver 116, and an antenna 118. The transducer array 112 emits ultrasound signals (waves) towards the subject 105 and receives ultrasound echo signals (echoes) reflected from the interior of the subject 105 back to the transducer array 112. The transducer array 112 may be coupled to a microbeamformer (not shown), and controls reception of signals by the acoustic elements. In exemplary embodiments, the transducer array 112 may be a LX-dimensional array, such as a 1.25D array or a 1.5D array, for example. In other embodiments, the transducer array may be arranged in a one-dimensional (1D) array or in a two-dimensional (2D) array.

The transducer array 112 includes an array of acoustic elements, which may be referred to as transducer elements. The transducer elements may be capacitive micromachined ultrasonic transducers (CMUTs) or piezoelectric transducers formed of materials such as PZT or PVDF, for example. Each transducer element may emit ultrasound signals into the subject 105 and receive ultrasound echo signals as the ultrasound signals are reflected from within the subject 105. The transducer elements generate analog electrical signals representative of the received ultrasound echo signals. The transducer array 112 may include M transducer elements arranged in row(s) and column(s) producing M analog ultrasound echo signals.

The probe circuitry 114 positioned within the ultrasound scanner 110 may be of any suitable type of circuitry and may serve several functions. For example, the probe circuitry 114 may include analog front-ends (AFEs) and analog-to-digital converters (ADCs) for conditioning and digitizing analog electrical signals output by the transducer elements of the transducer array 112. The probe circuitry 114 may further include multiplexers (MUXs), encoders and serializers for multiplexing, encoding and serializing the digitized electrical signals output by the ADCs, respectively, to provide serial digital data streams to the transceiver 116. The multiplexers may receive the digitized electrical signals over M signal lines and output multiplexed digitized electrical signals over L signal lines, where L is less than M. The probe circuitry 114 may include various other components such as resistors, capacitors, transistors, inductors, relays, clocks, timers, or any other suitable electrical components that may be integrated in an integrated circuit. In various configurations, the probe circuitry 114 may include hardware components, software components, and/or a combination of hardware components and software components, without departing from the scope of the present teachings.

The transceiver 116 is coupled to the probe circuitry 114 (via L signal lines) and the antenna 118. The transceiver 116 is configured to transmit the serial digital data streams to the host system 140 over the wireless communication link 130 through the antenna 118, and to receive control signals from the host system 140 over the wireless communication link 130 through the antenna 118 for controlling the ultrasound signals output by the transducer array 112. For example, the host system 140 may generate and transmit the control signals for controlling excitations of the transducer elements at the transducer array 112.

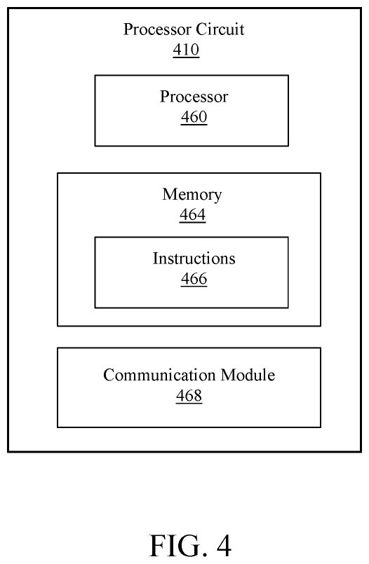

In addition, the ultrasound scanner 110 includes a processor circuit 120, an interface 122 and the display 125, discussed above. The processor circuit 120 is arranged within the housing 111 of the ultrasound scanner 110, and is described in more detail with reference to FIG. 4. The processor circuit 120 is configured to obtain at least one parameter regarding the ultrasound scanner 110, the subject 105, and/or the ultrasound imaging process. The at least one parameter may be received by the processor circuit 120 from the interface 122 and/or the transceiver 116, or the at least one parameter may be calculated by the processor circuit 120. The processor circuit 120 is configured to determine the content of the QR code 128 based on the at least one parameter, and to generate the QR code 128 such that it contains the determined content. The at least one parameter obtained by the processor circuit 120 may change to reflect different circumstances, so the content of the QR code 128 derived from the at least one parameter by the processor circuit 120 is variable.

As discussed above, the QR code 128 may include configuration information for establishing wireless communication between the ultrasound scanner 110 and the host system 140 over of the wireless communication link 130, for example. In this case, when the wireless communication link 130 is in a WiFi network, the parameters received by the processor circuit 120 may include a wireless IP address dynamically assigned by the WLAN, an SSID, a password, and/or an authentication data packet of the ultrasound scanner 110. The processor circuit 120 receives these parameters through the interface 122, e.g., from the user or from the WLAN itself as conveyed by the transceiver 116. For example, WiFi circuitry in the transceiver 116 may provide the assigned SSID, which is associated with a dynamically assigned IP address. The processor circuit 120 determines the content of the QR code 128 based on the received parameters, for example, by selecting and/or prioritizing the received parameters, calculating additional data using the received parameters, and/or deriving additional data from the parameters. The processor circuit 120 then generates the QR code 128 to show (include) the determined content. Techniques for generating QR code to show various contents are apparent to one skilled in the art.

In addition, or alternatively, a wide variety of other information may be contained in the QR code 128 based other types of parameters received by the processor circuit 120, respectively. For example, the at least one parameter may include identification information for identifying the ultrasound scanner 110, patient information for identifying the subject 105 and describing their condition, and/or procedure information for identifying the type of ultrasound procedure being performed. For example, the patient information may include the patient's medical record number (MRN) or other identifier. The processor circuit 120 receives these parameters, determines the corresponding content of the QR code 128, and then generates the QR code 128 to include this content. The at least one parameter may also include a location of the ultrasound scanner 110 (e.g., the procedure room) and/or at least one capability of the ultrasound scanner 110. In an embodiment, the ultrasound scanner 110 may also include a QR scanner (not shown) coupled to the processor circuit 120 through the interface 122. This QR scanner may be used to scan other QR codes, e.g., on the subject 105, to provide parameters to the processor circuit 120 to be incorporated into the contents of the QR code 128. Handling the wide variety of information is possible, at least in part, due to the dynamic generation and variability of the content included in the QR code 128.

The QR code 128 may also communicate sensitive data to the host system 140. For example, when the ultrasound scanner 110 collects sideband data other than the ultrasound images, such as patient data and/or a region of interest on the patient's body shown in photos (e.g., if the ultrasound scanner 110 includes a camera or an interface to a camera), then the ultrasound scanner 110 will contain especially sensitive information. The processor circuit 120 may receive the sensitive data as the parameters, generate a secure summary of the sensitive data as the content, and generate the QR code 128 to include the content. Reading the QR code 128 allows the host system 140 to confirm whether it should access the ultrasound scanner 110 and/or the sensitive data before an actual wireless connection is established between the ultrasound scanner 110 and the host system 140. For example, the host system 140 may confirm and/or record the secure summary of the sensitive data in the QR code 128 before establishing the actual wireless connection with the ultrasound scanner 110. Similarly, host system 140 may confirm and/or record the secure information about the ultrasound scanner 110 itself before establishing the actual wireless connection with the ultrasound scanner 110. Also, the host system 140 may authenticate the connection by unencrypting a password in the QR code 128 using a proprietary algorithm combined with the rest of the content in the QR code 128. The variable content in the QR code 128 therefore provides fast, authenticated, reliable selection of ultrasound scanners, as well as secure identification for dynamic, sensitive data.

The interface 122 interfaces the processor circuit 120 with the user and/or with another system, network or database. The interface 122 supplies the at least one parameter to the processor circuit 120, and/or supplies information from which the processor circuit 120 is able to determine the at least one parameter. When the interface 122 is a user interface, it may include a touchpad, a touchscreen, voice or gesture recognition captured by a microphone or video camera, a joystick, a mouse, a keyboard, or a trackball, for example. All or part of the user interface may be incorporated with the display 125 as a graphical user interface (GUI) for displaying and receiving information to and from the user. When the interface 122 is a network interface, it may include one or more of ports, disk drives, wireless antennas, or other types of receiver circuitry, for example.

FIG. 3 is a simplified block diagram of the host system in the ultrasound imaging system, according to a representative embodiment.

Referring to FIG. 3, the host system 140 may be any suitable computing and display device, such as a workstation, a personal computer (PC), a laptop, a tablet, a mobile phone, or a patient monitor, for example. In the depicted configuration, the host system 140 includes an antenna 148, a transceiver 146, host circuitry 144, and the host display 155. The transceiver 146 is configured to receive the serial digital data streams from the ultrasound scanner 110 over the wireless communication link 130 through the antenna 148, and to transmit control signals to the ultrasound scanner 110 over the wireless communication link 130 through the antenna 118 for controlling the ultrasound signals output by the transducer array 112.

The host circuitry 144 may be configured to support the digital signals transmitted to or from the ultrasound scanner 110, including the digital data streams received from the ultrasound scanner 110 over the wireless communication link 130. The host circuitry 144 within the host system 140 may be of any suitable type of circuitry and may serve any suitable functions. For example, the host circuitry 144 may include de-serializers, decoders and de-multiplexers (DEMUXs) for respectively de-serializing, decoding and de-multiplexing the serial digital data output by the transceiver 146, thereby expanding the L signal lines received from the ultrasound scanner 110 to the original M signal lines corresponding to the specific transducer elements or groups or patches of transducer elements within the transducer array 112. The host circuitry 144 may further include various other components such as resistors, capacitors, transistors, inductors, relays, clocks, timers, or any other suitable electrical components that may be integrated in an integrated circuit. In various configurations, the host circuitry 144 may include hardware components, software components, and/or a combination of hardware components and software components, without departing from the scope of the present teachings.

The host circuitry 144 may be further configured to generate image signals for display to a user on the host display 155 and/or perform image processing and image analysis for various diagnostic modalities or ultrasound types (B mode, CW Doppler, etc.). The circuitry 134 may additionally include one or more processing circuits, an example of which is discussed below with reference to FIG. 4. For example, the one or more processing circuits may include a general purpose computer, a computer processor, a microprocessor, a graphics processing unit (GPU), a central processing unit (CPU), a digital signal processor (DSP), a microcontroller, a state machine, programmable logic device, field programmable gate arrays (FPGAs), application specific integrated circuits (ASICs), systems on a chip (SOC), or combinations thereof. The one or more processing circuits may be configured to generate image signals from the de-multiplexed digital echo data streams and/or perform image processing and image analysis for various diagnostic modalities.

The host display 155 is coupled to the host circuitry 144. The host display 155 may include a monitor, a touch-screen, a television, an LCD, an LED display, a flat panel display, a solid-state display, a cathode ray tube (CRT) display, or any suitable display, for example, although any type of compatible display may be incorporated without departing from the scope of the present teachings. The host display 155 is configured to display images and/or diagnostic results processed by the host circuitry 144.

In addition, the host system 140 includes a processor circuit 150, an interface 152 and the QR scanner 145, discussed above. The processor circuit 150 may be substantially the same as the processor circuit 120 as described in more detail with reference to FIG. 4. The QR scanner 145 is configured to read the QR code 128 displayed on the display 125 of the ultrasound scanner 110. The processor circuit 150 is configured to receive the content of the QR code 128, and to extract the information contained therein. For example, the content of the QR code may indicate configuration information for establishing wireless communication with the ultrasound scanner, as discussed above. The processor circuit 150 may provide the information to the user and/or to another system, network or database via the interface 152 and/or the host display 155. When the interface 152 is a user interface, it may include a touchpad, a touchscreen, voice or gesture recognition captured by a microphone or video camera, a joystick, a mouse, a keyboard, or a trackball, for example. All or part of the user interface may be incorporated with the host display 155 as a GUI for displaying and receiving information to and from the user. When the interface 152 is a network interface, it may include one or more of ports, disk drives, wireless antennas, or other types of receiver circuitry, for example.

In an embodiment, the processor circuit 120 may include digital signal processing required to generate full ultrasound images from the digitized signals output by the transducer array 112. In this case, the display 125 of the ultrasound scanner 110 also may be configured to display the ultrasound images and/or diagnostic results processed at the ultrasound scanner 110 itself. That is, the processor circuit 120 (or one or more separate dedicated processor circuits) would be configured to generate image signals from the digital data output by the ADCs, for example, and/or to perform image processing and image analysis for various diagnostic modalities. There would be no need to create and send the digital echo data stream over the wireless communication link 130 to the host system 140 for processing and image display, although doing so is still an option if desired by the user. This affords a very large reduction in data bandwidth of the wireless communication link 130. The digital signal processing may include, for example, radio frequency (RF) filtering, time-gain compensation (TGC), analytic envelope detection, Doppler detection, arithmetic logging, post-detection filtering, decimation, compression, insertion of timing data, and the like.

FIG. 4 is a simplified schematic diagram of a representative processor circuit implemented by the ultrasound imaging system 100, according to a representative embodiment. One or more processor circuits 410 may be configured to carry out the operations described herein. The processor circuit 120 and/or any processor circuit in the probe circuitry 114 of the ultrasound scanner 110 may be implemented as the processor circuit 410. Likewise, the processor circuit 150 and/or any processor circuit in the host circuitry 144 of the host system 140 may be implemented as the processor circuit 410.

Referring to FIG. 4, the processor circuit 410 includes a processor 460, memory 464, and a communication module 468. These elements may be in direct or indirect communication with each other, for example via one or more buses.

The processor 460 may be implemented by a general purpose computer, a computer processor, a microprocessor, a GPU, a CPU, a DSP, a microcontroller, a state machine, programmable logic device, FPGAs, ASICs, SOCs, or combinations thereof, using any combination of hardware, software, firmware, hard-wired logic circuits, or combinations thereof. Additionally, any processing unit or processor herein may include multiple processors, parallel processors, or both. Multiple processors may be included in, or coupled to, a single device or multiple devices.

The term “processor” as used herein encompasses an electronic component able to execute a program or machine executable instruction. References to a computing device comprising “a processor” should be interpreted to include more than one processor or processing core, as in a multi-core processor. A processor may also refer to a collection of processors within a single computer system or distributed among multiple computer systems, such as in a cloud-based or other multi-site application. The term computing device should also be interpreted to include a collection or network of computing devices each including a processor or processors. Programs have software instructions performed by one or multiple processors that may be within the same computing device or which may be distributed across multiple computing devices.

The memory 464 stores instructions executable by the processor 460. The memory 464 may include a main memory and/or a static memory, where such memories may communicate with each other and the processor 460 via one or more buses. The memory 464 stores instructions used to implement some or all aspects of methods and processes described herein. The memory 464 may include a cache memory (e.g., a cache memory of the processor 460), random access memory (RAM), magnetoresistive RAM (MRAM), read-only memory (ROM), programmable read-only memory (PROM), erasable programmable read only memory (EPROM), electrically erasable programmable read only memory (EEPROM), flash memory, solid state memory device, hard disk drives, other forms of volatile and non-volatile memory, or a combination of different types of memory.

The memory 464 includes a non-transitory computer-readable medium that stores instructions 466. The instructions 466 may include instructions that, when executed by the processor 460, cause the processor 460 to perform the operations described herein with reference to the ultrasound scanner 110 and/or the host system 140. The instructions 466 may also be referred to as code. The terms “instructions” and “code” should be interpreted broadly to include any type of computer-readable statement(s). For example, the terms “instructions” and “code” may refer to one or more programs, routines, sub-routines, functions, procedures, etc. “Instructions” and “code” may include a single computer-readable statement or many computer-readable statements. As used herein, the term “non-transitory” is to be interpreted not as an eternal characteristic of a state, but as a characteristic of a state that will last for a period. The term “non-transitory” specifically disavows fleeting characteristics such as characteristics of a carrier wave or signal or other forms that exist only transitorily in any place at any time. The memory 464 may be secure and/or encrypted, or unsecure and/or unencrypted.

The communication module 468 may include any electronic circuitry and/or logic circuitry to facilitate direct or indirect communication of data between the processor circuit 120, the interface 122 and the display 125 in the ultrasound scanner 110, and/or between the processor circuit 150, the interface 152, the QR scanner 145, and the host display 155 in the host system 140. In that regard, the communication module 468 may be an input/output (I/O) device. In some instances, the communication module 468 facilitates direct or indirect communication between various elements of the ultrasound scanner 110 and the host system 140.

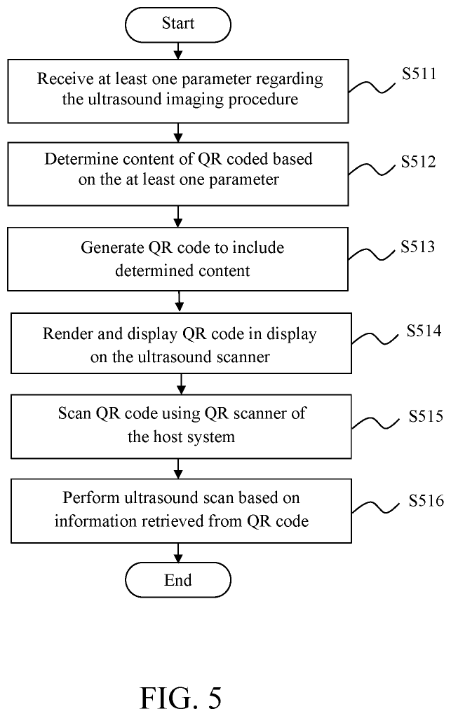

FIG. 5 is a flow diagram of a method of connecting a wireless ultrasound scanner with a host system, according to a representative embodiment. The method may be implemented, for example, using the processor circuit 120 in the ultrasound scanner 110 and the processor circuit 150 in the host system 140, described above.

Referring to FIG. 5, at least one parameter is received in block S511. The at least one parameter provides information related to the ultrasound imaging to be performed by the ultrasound scanner and the host system. For example, the at least one parameter may provide information enabling configuration of the ultrasound scanner and/or the host system for establishing communication over the wireless network. The at least one parameter may also provide any additional information that improves efficiency and security of the ultrasound imaging system and procedure, including identifying the ultrasound scanner, the patient, the procedure, and security criteria, such as passwords and authentication data, for example.

In block S512, content of a QR code is determined based on the at least one parameter. The content of the QR code is variable in that it may be determined in response to current information or factors that change over time. The content of the QR code may be determined in real time to adjust for changes in the ultrasound imaging procedure, such as reconnecting the wireless network, changing patients, starting a new imaging procedure, and the like.

In block S513, the QR code itself is generated to include the determined content. Since the content that the QR code contains is variable, the QR code likewise is variable and is re-generated each time the corresponding content changes.

In block S514, the QR code is rendering and displayed in a display on the ultrasound scanner. The display may be an LCD, an LED display, a flat panel display, or a solid-state display, for example, that is arranged on the housing of the ultrasound scanner, for example.

In block S515, the QR code is scanned using a QR scanner of the host system. The QR scanner may be a camera, for example, integrated with or otherwise attached to the host system. The scanning provides the host system the information encoded in the content of the QR code.

In block S516, ultrasound imaging is performed on the patient using the ultrasound scanner in association with the host system based at least in part on the information retrieved from the QR code by the host system. The scanning includes transmitting ultrasound signals into the patient from a transducer array in the ultrasound scanner, receiving corresponding ultrasound echo signals, and outputting corresponding electrical echo signals. The electrical echo signals are digitized, and may be transmitted to the host system over the wireless network. The host system processes the digitized echo signals for display. Alternatively, the ultrasound scanner may be configured to process the digitized echo signals for display, in which case image data as opposed to digitized echo signals may be transmitted to the host system over the wireless network. In this case, the image data may be displayed at the ultrasound scanner (e.g., in the same display that showed the QR code), at the host system, or both.

Thus, according to various embodiments, a handheld ultrasound scanner (e.g., ultrasound probe) is configured to present a variable QR code that can be read by a partnered host system. The QR code is rendered directly on a display on the housing of the ultrasound scanner, and is variable because it is responsive to changing conditions, including in real time. For example, the QR code may be used to automate a wireless connection between the ultrasound scanner and the host system even when the association mechanism is controlled dynamically by network infrastructure. Additional content of the variable QR code may include sensitive data, such as patient MRN, when the ultrasound scanner collects sideband data beyond ultrasound images. A secure summary of the sensitive data also may be in the QR code, allowing the host system to confirm or record the secure summary before the actual wireless connection is established. Accordingly, the various embodiments provide reliable, efficient association and connection of a wireless ultrasound scanner to its host system, confidence that the correct ultrasound scanner has been selected, confidence that the correct patient being scanned, and that auxiliary information regarding the ultrasound scanner and/or the patient is securely transferred before the connection is made between the ultrasound scanner and the host system.

Although methods, systems and components for implementing imaging protocols have been described with reference to several exemplary embodiments, it is understood that the words that have been used are words of description and illustration, rather than words of limitation. Changes may be made within the purview of the appended claims, as presently stated and as amended, without departing from the scope and spirit of the protocol implementation of the present teachings. The preceding description of the disclosed embodiments is provided to enable any person skilled in the art to practice the concepts described in the present disclosure. As such, the above disclosed subject matter is to be considered illustrative, and not restrictive, and the appended claims are intended to cover all such modifications, enhancements, and other embodiments which fall within the true spirit and scope of the present disclosure. Thus, to the maximum extent allowed by law, the scope of the present disclosure is to be determined by the broadest permissible interpretation of the following claims and their equivalents and shall not be restricted or limited by the foregoing detailed description.

The Abstract of the Disclosure is provided to comply with 37 C.F.R. § 1.72(b) and is submitted with the understanding that it will not be used to interpret or limit the scope or meaning of the claims. In addition, in the foregoing Detailed Description, various features may be grouped together or described in a single embodiment for the purpose of streamlining the disclosure. This disclosure is not to be interpreted as reflecting an intention that the claimed embodiments require more features than are expressly recited in each claim. Rather, as the following claims reflect, inventive subject matter may be directed to less than all of the features of any of the disclosed embodiments. Thus, the following claims are incorporated into the Detailed Description, with each claim standing on its own as defining separately claimed subject matter.

1. An ultrasound scanner for acquiring ultrasound images of a subject, the ultrasound scanner comprising:
a handheld housing;
a transducer array arranged within the housing, wherein the transducer array comprises a plurality of transducer elements configured to emit ultrasound signals into the subject, and to receive ultrasound echo signals responsive to the ultrasound signals being reflected in the subject;
at least one processor arranged within the housing, wherein the at least one processor is configured to obtain at least one parameter, determine content of a quick response (QR) code based on the at least one parameter, and to generate the QR code, wherein the content of the QR code is variable; and
a display arranged on the housing, wherein the display is configured to render and display the QR code generated by the at least one processor, wherein the QR code is readable by a QR scanner of a host system.

2. The ultrasound scanner of claim 1, further comprising:
a transceiver configured to establish wireless communication with the host system via a wireless communication link,
wherein the at least one parameter comprises configuration information of the wireless communication link for establishing the wireless communication, and
wherein the content of the QR code includes the configuration information from the at least one parameter.

3. The ultrasound scanner of claim 2, wherein the wireless communication link comprises a WiFi link, and wherein the configuration information comprises a service set identifier (SSID), a wireless IP address, a password, and/or an authentication data packet of the ultrasound scanner.

4. The ultrasound scanner of claim 2, wherein the at least one parameter comprises a secure summary of personal data of the subject to be included in the content of the QR code, wherein the host system confirms and/or records the personal data by reading the QR code prior to establishing the wireless communication with the transceiver of the ultrasound scanner.

5. The ultrasound scanner of claim 1, wherein the at least one parameter comprises a unique identifier identifying the ultrasound scanner, a unique identifier identifying the subject, and/or secure encoding of a condition of the subject to be included in the content of the QR code.

6. The ultrasound scanner of claim 1, wherein the at least one parameter comprises a location of the ultrasound scanner and/or at least one capability of the ultrasound scanner to be included in the content of the QR code.

7. The ultrasound scanner of claim 1, wherein the at least one processor comprises an ultrasound image processor configured to generate ultrasound image data corresponding to the ultrasound echo signals; and
wherein the display is further configured to display at least one ultrasound image based on the ultrasound image data from the image processor.

8. The ultrasound scanner of claim 1, further comprising:
a camera configured to acquire at least one image of the subject, and to provide corresponding image data to the at least one processor.

9. The ultrasound scanner of claim 8, wherein the at least one processor is further configured to determine content of the QR code based on the image data.

10. The ultrasound scanner of claim 1, wherein the at least one parameter comprises encoded content used for authentication of the ultrasound scanner by the host system and for secure identification of sensitive data provided by the ultrasound scanner.

11. The ultrasound scanner of claim 1, wherein the display comprises a liquid crystal display (LCD), a light emitting diode (LED) display, a flat panel display, or a solid-state display.

12. A method of connecting a wireless ultrasound scanner with a host system for performing ultrasound imaging of a subject, the method comprising:
receiving at least one parameter providing information related to the ultrasound imaging;
determining content of a quick response (QR) code based on the at least one parameter, wherein the content of the QR code is variable;
generating the QR code to include the determined content;
rendering and displaying the QR code in a display arranged on the ultrasound scanner; and
reading the QR code at the host system using a QR scanner to retrieve the content of the QR code, enabling performance of the ultrasound imaging of the subject.

13. The method of claim 12, wherein the at least one parameter provides configuration information enabling configuration of the ultrasound scanner and/or the host system for establishing communication over the wireless network.

14. The method of claim 13, wherein the wireless network comprises a WiFi link, and wherein the configuration information comprises a service set identifier (SSID), a wireless IP address, a password, and/or authentication data of the ultrasound scanner.

15. The method of claim 12, wherein the at least one parameter provides information identifying the ultrasound scanner, the patient, a procedure, and/or security criteria.

16. The method of claim 12, wherein the content of the QR code is determined in real time to adjust for changes in a procedure for performing the ultrasound imaging.

17. The method of claim 12, further comprising:
performing the ultrasound imaging of the subject using the ultrasound scanner in association with the host system based at least in part on the information in the QR code read by the host system.

18. The method of claim 17, further comprising:
displaying an ultrasound image resulting from the ultrasound imaging of the subject on the display arranged on the ultrasound scanner.

19. An ultrasound scanner for acquiring ultrasound images of a subject, the ultrasound scanner comprising:
a handheld housing;
a transducer array arranged within the housing, wherein the transducer array comprises a plurality of transducer elements configured to emit ultrasound signals into the subject, and to receive ultrasound echo signals responsive to the ultrasound signals being reflected in the subject;
a transceiver configured to establish wireless communication with a host system via a wireless communication link;
at least one processor arranged within the housing, wherein the at least one processor is configured to obtain configuration information of the wireless communication link for establishing wireless communication with the host system, to determine content of a quick response (QR) code based on the configuration information, and to generate the QR code, wherein the content of the QR code is variable; and
a display arranged on the housing, wherein the display is configured to render and display the QR code generated by the at least one processor, wherein the QR code is readable by a QR scanner of the host system to establish the wireless communication.

20. The ultrasound scanner of claim 19, wherein the wireless communication link comprises a WiFi link, and wherein the configuration information comprises a service set identifier (SSID), a wireless IP address, a password, and/or an authentication data packet of the ultrasound scanner.
